(19) United States
(12) Patent Application Publication
(10) Pub. No.: US 2008/0268508 A1
(43) Pub. Date: Oct. 30, 2008

(54) Methods and kits for negative selection of desired nucleic acid sequences

(76) Inventor: Mohankumar R. Sowlay, Phillipsburg, NJ (US)

Correspondence Address: Mohankumar R. Sowlay, 13 Melanie Lane, Phillipsburg, NJ, 08865, US

(21) Appl. No.: 11/796,983

(22) Filed: Apr. 30, 2007

(51) Int. Cl. C12P 19/34; C07H 21/00; C12N 9/12

(52) U.S. Cl. 435/91.2; 435/194; 435/262; 435/267; 435/268; 536/23.1; 536/25.4

(57) ABSTRACT

The present invention pertains to a method to isolate, separate, enrich or amplify a targeted nucleotide polymer such as mRNA through selective reverse transcription of the targeted polymer into cDNA from a sample comprising of chemically identical or similar polynucleotide polymers such as rRNA. The enrichment of the targeted nucleic acid such as mRNA is accomplished by blocking the reverse transcription of undesired rRNA while allowing unrestricted reverse transcription of the targeted polymer. The invention also embodies that the cleavage of the non-targeted nucleic acid such as rRNA bound to an oligonucleotide through enzymatic activity (RNase H). The invention further embodies methods and kits to accomplish the utility of the invention through the following steps 1) 3′ tailing of chemically identical or similar nucleotide polymers in a sample that includes bacterial mRNA 2) a 3′ tail capable of binding to a oligo-dN primer 3) at least one oligonucleotide capable of preventing the extension of oligo-dN bound to at least one non-targeted nucleotide polymers by a DNA polymerase such as a reverse transcriptase without restricting conversion of bacterial mRNA into cDNA 4) where the non-targeted molecule is prevented as a template for cDNA synthesis by enzymatic cleavage (RNase H) of template (rRNA)-oligonucleotide hybrid 5) where the reverse transcriptase is physically blocked by the oligonucleotide bound to the non-targeted nucleic acids such as rRNA 5) purification of the selectively transcribed cDNA. In further embodiments of the present invention, methods and composition to enable the study of bacterial transcriptomics-an analysis of genes expressed by a bacterial infection of a host, an isolated bacterial culture or a bacterial community, such as recovered from soil, intestine, mouth, biofilm, water etc are also included for use in DNA-chip or sequencing analyses.

### FIELD OF THE INVENTION

This invention discloses methods to enrich bacterial mRNA in its native form or through its direct conversion into complementary DNA. More specifically, the methods use species-specific or universal probes that can hybridize to bacterial or eukaryotic rRNA and other RNAs such as tRNA, small nuclear RNA or other nucleic acid molecules etc. The invention also relates to the use of modified or unmodified oligonucleotide probes that are either 1) derivatized with magnetic beads, 2) non-extendable in the presence of a polymerase, its template and nucleotides, or 3) covalently linked to an RNase H moiety. The invention further uses RNase H in conjunction with oligonucleotide probes that can selectively destroy RNA targets.

### BACKGROUND TO THE INVENTION

Bacterial functional genomics involves the study of all genes expressed in the form of messenger RNA (mRNA) transcripts by a bacterial culture in the laboratory or a bacterial community adapted to an ecological setting in nature. The importance of these mRNA transcripts is that they form informational templates for making functional molecules such as proteins, many of which are enzymes that mediate cellular activities. Very often tackling multi-drug or antibiotic resistance in infectious bacteria requires identification of certain protein or a nucleic acid molecule as potential drug targets. An understanding of how bacterial expression is controlled and how their proteins might function has not only academic but also industrial relevance. Typically, enzymatic intervention in as diverse activities as degrading food or enhancing its flavor, transforming hydrocarbon compounds or causing infection has the potential to unleash biotechnological products with agricultural, gastronomical, environmental or medical applications.

Unfortunately, in spite of the remarkable strides witnessed in modern biotechnology in the last couple of decades, one prize for the practitioners of molecular microbiology that has remained elusive is the ability to convert the entire bacterial mRNA expressed at any given moment into their stable complementary DNA (cDNA) counterparts. Current evidence shows that prior to the identification and isolation of individual prokaryotic mRNA transcript, molecular cloning of a gene, such as a gene coding for protein A from Staphylococcus aureus, was a routine technique which enabled establishing its gene sequence and function. Using the gene sequence, fluorescent, digoxigenin or radio-labeled gene probes were used to detect individual mRNA transcripts in RNA samples obtained under different experimental or environmental conditions. For instance, digoxigenin labeled transcript probes were used to detect a virulence mRNA in Listeria monocytogenes cells. However, a particularly daunting technical challenge presented by almost all of bacterial mRNA is the lack of a signature sequence in the form of polyadenylated tail on the bacterial mRNA transcripts that would enable the capture of all bacterial mRNA transcripts similar to the eukaryotic mRNA. Besides, targeted capture of bacterial mRNA is difficult since they are chemically identical to ribosomal RNA (rRNA) that are present in abundance at more than 98% of the total cellular RNA pool.

Nevertheless, a number of attempts have been made previously to recover total bacterial mRNA. In some of the studies conducted in early to mid 1980's, intact polyadenylated tails of Bacillus subtilis mRNA were targeted for initiating oligo dT mediated cDNA synthesis. However, this facile strategy was not successful owing to the fact that the only 2 to 60% of the transcripts were associated with variable and very short 3′ poly A tracts. For instance, in a recent scientific study, it was reported that oligo-dT priming of mycobacterial mRNA was unsuccessful in yielding a representative sample of cDNA due to inadequately polyadenylated mRNA, thereby frustrating our ability to obtain useful insights into its gene expression under different experimental conditions and thus understand its physiology. In a yet another study recently, a multi-step technique designed to isolate total mRNA from Escherichia coli through selective polyadenylation of polysome bound mRNA using Poly A Polymerase and, magnesium and manganese divalent cations, predicated upon successful polysome isolation, resulted in polyadenylation of about half the mRNA molecules. Besides, the technique did not ensure exclusion of rRNA from polyadenylation and subsequent oligo-dT mediated reverse transcription of these molecules.

Among the methods to reduce or even eliminate the preponderance of rRNA molecules is the specific subtraction of rRNA using biotin labeled antisense rRNA derived from plasmid borne and Polymerase Chain Reaction (PCR) amplified Staphylococcus aureus rRNA gene fragment. Another approach employed to determine the differentially expressed prokaryotic mRNA is the subtraction of not only the rRNA but also house-keeping and structural genes isolated from treated bacterial cultures by hybridizing with cDNA made from total RNA of untreated cultures. Although these techniques are useful, they are neither simple nor rapid to enrich bacterial mRNA or convert the transcripts into cDNA for routine applications such as DNA-DNA hybridization assays or nucleic acid sequencing.

Profound interest in bacterial functional genomics has been aroused in industrial research groups as a result of the discovery of novel biocatalysts from marine microorganisms including enzymes for cleaning agents that are active at low temperatures, or food applications. However, attempts to recover total bacterial transcripts to screen for similarly unusual and useful properties of genes and gene products expressed in response to environmental stimulons from various ecologically adapted microbial communities have not been particularly rewarding due to inefficient isolation and screening techniques and presumed short shelf life of mRNA. A further complication in the recovery of novel gene transcripts is neither the natural habitats of these microorganisms can be successfully replicated nor do suitable synthetic media developed to cultivate those microorganisms in a laboratory. Therefore, a novel approach was adopted to recover mRNA transcripts from pure cultures of Pseudomonas putida and soil microorganisms. The differential display technique was improvised by using poly (T) primers and a modified Shine-Dalgarno sequence (signaling sequence for bacterial protein biosynthesis) specific primer to amplify the expressed sequences. However, lack of uniformly polyadenylated mRNAs and highly variable Shine-Dalgarno sequences, or lack of such a sequence, give rise to unacceptably high false positives thereby limiting the utility of this approach.

Several other strategies have been devised to circumvent the nearly intractable problem of recovering total bacterial mRNA transcripts. Breakthroughs in genomic technologies have spawned a number of alternative approaches to decipher bacterial gene function. Methods such as cloning meta-genomic libraries of environmental bacterial genomes for natural products discovery in drug development or for screening novel enzymes for biocatalysis with comparative DNA sequencing analysis to identify genes are insufficient without understanding the context or extent of their expression. In the last decade, advances in automated DNA sequencing has lead to rapid sequencing of whole bacterial genome but at great cost, time and effort. A profusion of microbial genomic data from 235 microbial genomes sequenced until today and an array of bioinformatics tools created during the race to sequence human genome have aided and spurred development of appropriate tools for comparative genome analyses and identification of bacterial transcripts.

One of the latest approaches is to interrogate the immobilized putative gene fragments or their signature gene fragments identified through genome sequencing projects by using total bacterial RNA. A typical application is the oligonucleotide micro-array in monitoring gene expression of cultured bacteria such as Bacillus subtilis, Streptococcus pneumoniae or natural microbial populations. Again, the overwhelming preponderance of rRNA, nearly 25 to 50 times the numbers of mRNA by any typical total RNA extraction process, interfere mRNA binding and detection on these arrays. However, given the fact that nearly 90% of the bacteria are uncultivated or uncultivable in the laboratory, massive sequencing efforts, without considering bacterial gene expression and function in their natural habitats, is mere information without utility in terms of understanding gene function.

Finally, U.S. Application No. 20030175709 describes efforts to devise a simple mRNA enrichment technique. The technique employs rRNA probes with nucleotide tail that can bind both 16S and 23S rRNAs as well as a common oligonucleotide capture probe derivatized with paramagnetic beads with subsequent subtraction of these bound rRNAs on a magnetic stand allowing aspiration of mRNA in supernatant solution into a separate tube. The procedure described is similar to the repeat capture of Chlamydia trachomatis 16S rRNA by hybridization of an oligo-dT derivatized with magnetic beads to the poly-A tail of an oligo bound to the microbial16S rRNA. However, the procedure requires multiple hybridization with multiple probes besides repetitious separation of magnetic bead bound RNAs extending procedural time, as described in U.S. Pat. No. 6,812,341, to accomplish sufficiently low levels of rRNA in a sample. A recent technique described in the U.S. Pat. No. 6,242,189 reveals a methodology to isolate bacterial mRNA bound to polysomes that have been selectively polyadenylated using Poly A Polymerase and its subsequent oligo-dT based capture or transcription requires the isolation of polysomes. Yet another commercially available product is the mRNA-ONLY™ prokaryotic mRNA isolation kit from Epicenter Biosciences (Madison, Wis.) that selectively digests rRNAs by 5′-Phosphate-dependent Exonuclease, a processive 5′-3′ exonuclease, without affecting the mRNAs. In identifying the shortcomings of the existing methodologies, it is recognized in the art the need to directly transcribe the bacterial mRNAs into cDNA transcripts while simultaneously restricting the transcription of rRNAs followed by its elimination in the same procedure. Conversion of bacterial mRNA into cDNA has the advantage of increasing the integrity and stability of the molecules for long term archival at lower temperatures and for further applications such as cloning, sequencing and in the study of bacterial gene expression such as environmental transcriptomics to understand gene expression in natural microbial communities. Methods provided herein overcome the disadvantages found in prior art. Besides, procedures contemplated in the present invention reduce time, and or research materials to accomplish bacterial mRNA enrichment as well as its conversion to its cDNA counterparts.

### SUMMARY OF THE INVENTION

The present invention relates to the compositions and use of at least one species specific or universal rRNA specific oligonucleotide derivatized with magnetic bead capable of binding to bacterial rRNA such as 16S and 23S and/or eukaryotic rRNA such as 28S rRNA and including tRNAs, fragmented RNA molecules, small nuclear RNA and the like to capture non-targeted RNA molecules with the purpose of enriching bacterial mRNA. The oligonucleotide used may optionally be modified with phosphodiester, phosphorothioate or peptide bonds and 3′ terminally modified such as with a dideoxynucleotide to prevent its extension by a polymerase or derivatized with a magnetic bead at its 5′ end.

The invention also contemplates the use of non-extendable species-specific or universal rRNA oligonucleotide probe to selectively block oligo-dT initiated reverse transcription. More specifically, the non-extension of a rRNA specific oligonucleotide may be accomplished with terminal modification with a dideoxynucleotide or with a 3′ nucleotide tail of the probe that does not bind the template. Furthermore, the rRNA oligonucleotide probe is designed to bind downstream of oligo-dT or an oligo-dU binding site to impede reverse transcription. The invention envisages selective transcription since polyadenylated bacterial mRNA templates are reverse transcribed by oligo-dT primers without any hindrance of blocking oligonucleotides.

Also, the present invention provides methods to accomplish selective reverse transcription of bacterial mRNA by targeting specific cleavage of rRNA bound to rRNA specific oligodeoxynucleotide probes in the presence of RNase H enzyme.

In yet another embodiment, the present invention is directed to pharmaceutical and diagnostic kits for enriching bacterial mRNA in its native form or by converting them to cDNAs. The kits may comprise of at least one rRNA specific oligodeoxynucleotides probe, buffers, a reverse transcriptase, RNase H, oligo-dT primer, Poly A Polymerase or one or more combination of these components.

It is also within the scope of this invention to provide kits, where appropriate, of combinations of two or more species specific or universal rRNA oligonucleotide probes. It is further the object of the present invention to provide methods of enriching bacterial mRNA and/or converting bacterial mRNA directly into cDNA from a sample mixture that has bacterial mRNA and other nucleic acids and proteins etc. In yet another embodiment, the invention also contemplates the use of bacterial cDNAs thus obtained to be used in array based diagnostic applications.

### BRIEF DESCRIPTION OF THE FIGURES

The figures provide graphic representation of how the present invention can be utilized to enrich bacterial mRNA in its native form or by converting them into their complementary DNA.

FIG. 1 is a diagram describing the methodology of subtracting rRNA from a sample mixture containing bacterial mRNA and other nucleic acid sequences. The technique employs rRNA specific oligonucleotides derivatized with magnetic beads that hybridize to rRNA and the hybridization complex is separated to a side leaving the mRNA in solution which can be aspirated into a separate tube.

FIG. 2 describes the direct conversion of polyadenylated bacterial mRNA into complementary DNA by reverse transcription while selectively blocking such oligo-dT initiated transcription of rRNA using non-extendable rRNA specific oligonucleotides bound downstream of the initiation site of reverse transcription.

FIG. 3 is a graphical representation of conversion of polyadenylated bacterial mRNA into complementary DNA by reverse transcription while selectively blocking such oligo-dT initiated transcription of rRNA using non-extendable rRNA specific oligonucleotides bound downstream of the initiation site of reverse transcription that cleave the rRNA template in the presence of RNase H.

### DETAILED DESCRIPTION OF THE INVENTION

In comparison to the foregoing techniques reviewed above, the present invention provides a detailed and yet a simple methodology to not only enrich bacterial mRNA from total RNA but also synthesize cDNA libraries of bacterial mRNA transcripts obtained as part of total bacterial RNA from a bacterial pure culture or a natural microbial community. The present invention, as contemplated, not only reduces time but also accomplishes targeted unrestricted conversion of only bacterial mRNA into cDNA. The present invention, in further embodiments, provides for preferential cDNA synthesis from mRNA templates while restricting the conversion of bacterial rRNA into cDNA. For those skilled in the art, it is not beyond their capability to modify the method for alternative applications involving preferential extension of universal primers using select polynucleotide targets as templates while blocking the extension of primers on undesirable targets.

In the preferred embodiment of the present invention, the sample is described as the material consisting of total bacterial RNA with or without other eukaryotic polynucleotide sequences such as DNA or RNA in a dry or an aqueous suspension. It will be obvious to one skilled in the art that polynucleotide polymers from both bacterial and its eukaryotic host such as an animal, plant or human or tissue, organ or body fluid originating from such a host could be recovered during a single extraction process. In further embodiments, the sample for the purposes of this invention is recovered from a bacterial population or an isolate from air, water, soil etc, resident in its natural terrestrial or subterranean habitat and/or as a symbiont, adventitious or an infectious agent in plant, animal or human population or derived there from. In additional embodiments of this invention bacterial cells may even have been cultivated in a laboratory after isolating individual or multiple isolates from any of its native environment and then the RNA material obtained either as a cell lysate after breaking open the cells using either water, heat, chemicals and/or microwave or as a purified suspension using RNA isolation kits.

It is well established in the public domain, the procedures and materials needed to isolate total RNA including mRNA from microorganisms (25, 26 & 27). In some embodiments of the methods disclosed herein, a user of the present invention might choose to employ any of the published or commercially available eukaryotic and prokaryotic RNA isolation kits such as those offered for sale by Qiagen Inc. of Carlsbad, Calif. to obtain RNA fragments, especially large molecules, including mRNA bound or unbound to polysomes. In a particular feature of this invention is the recognition that small RNA such as tRNA, degraded RNA molecules and especially those that are less than 100 nucleotides in length are removed during total RNA isolation and/or during the bacterial mRNA and cDNA synthesis procedure described in this invention.

Where prokaryotic and eukaryotic polynucleotide polymers are co-extracted, the poly (A) tail of eukaryotic mRNA can be targeted with commercially available mRNA kits offered by Promega, Wis., Invitrogen, CA Qiagen, Calif. that make use of oligo-dT or oligo-dT derivatized with magnetic beads for cDNA synthesis or mRNA isolation. Separation of eukaryotic mRNA facilitates the creation of a separate library of cDNA molecules, distinct from the synthesis of cDNA library of bacterial mRNA while precluding the full length cDNA synthesis of other RNA species. In certain aspects of the present invention, the eukaryotic mRNA is not separated, it may also be selectively reverse transcribed along with prokaryotic mRNA by inhibiting cDNA synthesis of all rRNA molecules by employing the protocol described in this invention.

The sample, for the purposes of this invention, may have any composition comprising mostly of RNA molecules including bacterial mRNA isolated from the enrichment techniques described in the preceding review of what is known in the art. Since no enrichment may guarantee 100% elimination of bacterial rRNA, the present invention may be used as an additional protocol in further reducing the numbers of bacterial rRNA or eukaryotic rRNA in the sample as well as converting them into cDNA.

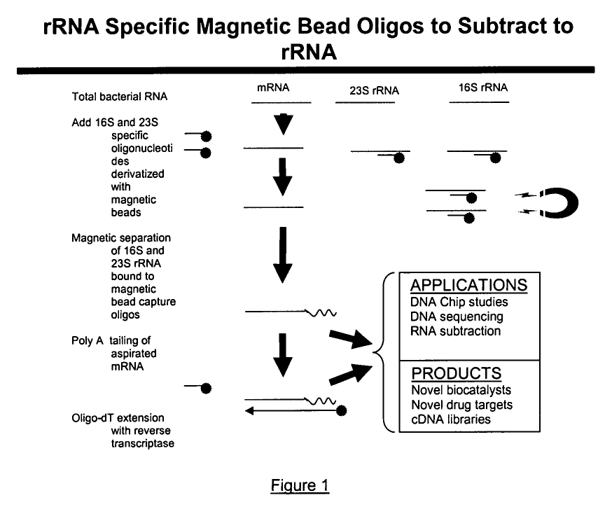

Multiple round enrichment of targeted nucleic acid molecule can be accomplished by hybridizing a biotinylated, gene specific oligonucleotide probe and the retrieval of the nucleic acid complex with paramagnetic streptavidin beads. A person skilled in the art may use streptavidin-coated magnetic beads bound to biotinylated rRNA specific oligonucleotide probes in targeting bacterial rRNA molecules. In a preferred embodiment of the present invention, at least two universal or species-specific, oligonucleotide sequences derivatized with magnetic beads, each on which is substantially complementary to bacterial ribosomal ribonucleic acid molecules, 16S rRNA and 23S rRNA, are specifically hybridized to them, in a sample mixture containing all RNA including bacterial mRNA (FIG. 1). Where sequences are complementary, other ribonucleic acid molecules such as eukaryotic RNA or 5S rRNA etc can also be targeted with sequence specific probes. Unlike the methodology described in US patent application No. 20030175709 no tailed nucleic acid probe to hybridize a target nucleic acid or an additional hybridization step with a magnetic bead derivatized oligonucleotide probe to capture the initial hybridization complex is contemplated in this invention. A simple one-step hybridization is followed by the physical separation of the hybridization complex or targeted rRNA and magnetic bead probes by means of a magnet to the side of a tube and the supernatant containing bacterial mRNA is aspirated into a separate tube. The procedure described in this invention to isolate all rRNA is similar to the technique of employed using rRNA capture probes to capture select, ¹³C labeled naturally occurring rRNA (30). However, the present invention envisions negative selection of the desired bacterial mRNA molecules by targeting most or all of the non-mRNA nucleic acid molecules with molar excess or substantially higher amounts or rRNA capture probes derivatized with magnetic beads followed by magnetic separation of capture probe and rRNA hybrids. The bacterial mRNA, thus enriched, can be used to synthesize cDNA employing standard molecular biology techniques known to one skilled in the art. Moreover, the strength of hybridization between capture oligonucleotide probe and its complementary target molecule can be altered by varying the temperature and adjusting the concentration of salts.

Instead of enriching bacterial mRNA prior to its conversion to cDNA, it may be desirable in certain applications to accomplish synthesis of cDNA from bacterial mRNA by selectively restricting the cDNA synthesis of rRNA, bacterial or eukaryotic thereby ensuring cDNA synthesis and enrichment of targeted nucleic acid molecules such bacterial mRNA.

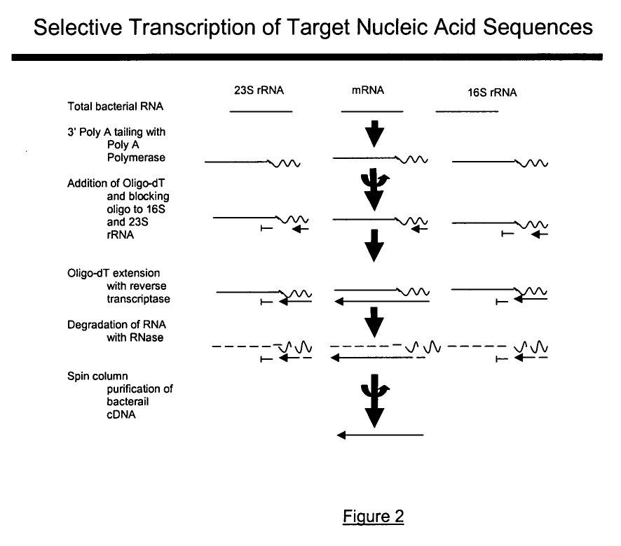

In a preferred embodiment of the present invention as delineated in FIG. 2 total bacterial RNA sample, comprising of mRNA and rRNAs etc, is subjected to uniform catalytic addition of adenosine residues to the 3′ tail of all RNA molecules to provide a poly A tail by incubating it in a reaction mixture comprising of Poly A polymerase (PAP), its substrate adenosine triphosphate (ATP) and appropriate buffer and reagents such as magnesium and sodium salt that are widely available in the marketplace. In a particular embodiment of this invention it is envisaged that a minimum of ten adenosine residues are added to the 3′ tail of RNA. The polyadenylation by the PAP occurs anywhere between the ambient temperature and 65 degrees Celsius. Where feasible, such polyadenylation step may be incorporated prior to concentrating or eluting the total RNA from a sample source comprising of soil samples from environment, body fluids, tissue or organ, a bacterial culture etc using any of the RNA isolation methods described in the public domain or through commercially available isolation kits. It is also intended as part of this invention that reagents for polyadenylation and/or cDNA synthesis could be supplied as part of a kit designed to convert bacterial mRNA into cDNA. Following the addition of the 3′ poly (A) tail to all RNA molecules, the enzyme, ATPs etc may be removed, if necessary, through spin column purification or through heat or chemical deactivation.

The present invention also includes enzymatic addition of homopolymeric or heteropolymeric nucleotide tracts using ATP, CTP, UTP, GTP or their mixtures and analogs.

The present invention also includes methods to enzymatically tail nucleotide sequences, for example, a technique to polyadenylate non-polyadenylated RNA such as bacterial mRNA by using PAP or to 3′ end label nucleotide triphosphates (NTP), deoxyNTP, dideoxyNTP, non-radioactive labels such as digoxigenin-11-UTP etc or add ATP, GTP and UTP homopolymeric tracts of various lengths. Generally, commercially available PAP isolated from Escherichia coli, yeast or mammalian sources or PAP obtained through recombinant DNA methods can be used for 3′ tailing of RNA. The enzyme terminal nucleotidyl transferase has also been used to add digoxigenin-11-UTP to RNA molecules. Yet another enzyme used to tail 3′ end of polynucleotide sequences is the polynucleotide phosphorylase and its homologs.

Methods to 3′ tail different classes of non-polyadenylated or insufficiently polyadenylated RNA molecules with nucleotides are also well known to one skilled in the art. Yeast PAP has been used to polyadenylate bacterial mRNA. In vitro RNA transcripts as well as Escherichia coli tRNA etc were 3′ end labeled using DIG- or biotin-dUTP, ddUTP or dATP and terminal nucleotidyl transferase. Total RNA recovered from environmental soil samples comprising of bacterial mRNA, 16S and 23S rRNA tRNA, fragments of RNA degraded during extraction process etc have all been successfully 3′ end tailed with poly (A) tail with PAP since they are all chemically indistinguishable templates for the PAP polyadenyaltion.

Although the U.S. Patent Application 0060051771 discloses a method for increasing the efficiency of 3′ tailing of RNA by heating the sample comprising of RNA molecules to at least 70 degrees Celsius for at least 10 minutes to alter the secondary structure of RNA, for the purposes of this invention, however, it is neither necessary to heat the sample to more than 65 degrees Celsius nor required to heat for at least 10 minutes at higher than 65 degree Celsius. Increase in the efficiency of poly (A) tailing with yeast PAP at the 3′ end of certain RNA species was observed at higher temperatures when tailing was performed over a temperature gradient of 25 to 60 degrees Celsius due to unfolding of RNA molecules. Therefore, in practicing this invention, it is not necessary to rely upon what is disclosed in US patent application 0060051771. In fact, increasing the temperature over 70 degrees Celsius imposes unnecessary complication and restriction in designing a mixture of oligonucleotides that have approximately similar melting temperature to bind to their complementary targets on the RNA and in having to modulate or use different temperature controlled heat blocks or water baths during the procedure. In accomplishing the 3′ tailing of all nucleic acid molecules in a sample, it is envisaged that the tail shall be at least 10 nucleotides in length.

The advent of molecular phylogeny, to study sequence similarity to discern common origin of molecules such as rRNA, and to understand the inter-relationships between bacteria and aid their classification has unleashed an explosion of 16S rRNA sequence data with over 253, 813 aligned sequences in the ribosomal database project. The profusion of sequencing data has been made possible due to the availability of bacterial rDNA probes and primers, which is by no means an exhaustive listing. The European ribosomal RNA database has over 13,500 and 1100 prokaryotic and over 6,500 and 150 eukaryotic small subunit (SSU) and large subunit (LSU) sequences respectively including a list of primers and probes. As additional bacterial 16s rDNA (SSU) and 23S rDNA (LSU) or eukaryotic 17S, 18S and 28S rDNA sequences become available, as it has, it allows designing improved primer and/or probes to hybridize to universally conserved sequences on the rRNA and tRNA molecules. In practicing the present invention modified and unmodified probe and primer sequences, established in public domain, is contemplated.

In further embodiments of this invention, the Poly A tailed rRNA molecules such as prokaryotic16S or 23S, eukaryotic 17S, 18s and 28S and tRNAs etc are targeted with a mixture of modified (non-extendable and/or chemical modification) or unmodified oligonucleotides which bind specifically to the universally conserved regions of these RNA molecules. The oligonucleotide binding region may preferably be that site of the rRNA molecules which are generally used for primer extension and/or rDNA probing purposes. In certain aspects of this invention, it is envisaged species-specific, genus-specific, taxa-specific rRNA molecules are hybridized with non-extendable oligonucleotides to block reverse transcription initiated upstream of their binding region. Simultaneously, oligo-dT, oligo-dU or oligo-dN primers are added depending upon the kind of 3′ tailing performed so that all tailed RNA molecules are targets for hybridization. The binding of these rRNA-specific oligonucleotides is anywhere on the rRNA template, preferably at the 3′ end of the rRNA molecules within a short distance of 250 bases or less, more preferably within 50 bases or less from the first adenosine residue of Poly A tail regions.

The present invention refers to modified oligos to include those, but not limited to, that a) may not have a 3′-OH group for a polymerase to use it as a primer for cDNA synthesis, b) have a 3′ tail of nucleotides that do not hybridize to the template RNA thus disabling cDNA synthesis from the bound oligo to the RNA template c) have a 5′ nucleotide tail that does ligate to another nucleic acid molecule d) are modified with phosphothioate (such as anti-sense oligonucleotides), phosphodiester (such as regular deoxyribonucleotides) or peptide bonds (such as Peptide Nucleic Acids) etc. e) are end-labeled with biotin, digoxigenin, radioactivity, dideoxynucleotides, fluorescent dyes 0 short oligonucleotides incapable of being extended by a polymerase etc. In additional embodiments of the present invention the added non-extendable oligonucleotides shall be at least 10 nucleotides in length.

In further embodiments of the present invention the added oligonucleotides, upon binding to the complementary target sites on 16S and 23S rRNA molecules only, selectively block the oligo-dT or oligo-dU or oligo-dN primed extension of the Poly A or Poly-N tailed rRNA molecules while allowing the extension of the oligo-dT or oligo-dN primer bound to the mRNA molecules in the presence of a DNA polymerase such as a reverse transcriptase (RT) enzyme. Where oligo-dU primer is used, as an initial step, reverse transcription could be carried out with dNTP mixture containing dUTP instead of dTTP. The rationale for doing so is to shorten the length of cDNA, especially from 23S rRNA, containing UTP using UNG glycosylase to facilitate removal rRNA cDNA during final spin column purification. Furthermore, following heat inactivation of UNG glycosylase, reverse transcription can be continued with regular dNTPs containing dTTPs.

Selective inhibition of reverse transcription initiated from primer oligo, by exogenously added oligonucleotides bound to RNA template, is well known to one skilled in the art. In the literature, the inhibition of the cDNA synthesis has been reported by the hybridization of oligonucleotides to RNA template about 100 nucleotides downstream of 3′ end of primer due to the endonucleolytic action of the RNase H activity associated with the AMV-RT or MMLV-RT that cleaves the RNA template bound to the oligo yielding truncated cDNA products. In yet another study, modified (phosphorothioate) and unmodified anti-sense oligodeoxyribonucleotides were shown to block reverse transcription initiated by Human Immuno-deficiency Virus Reverse Transcriptase (HIV-1 RT) by cleaving the RNA template bound to anti-sense oligo through RNase H activity.

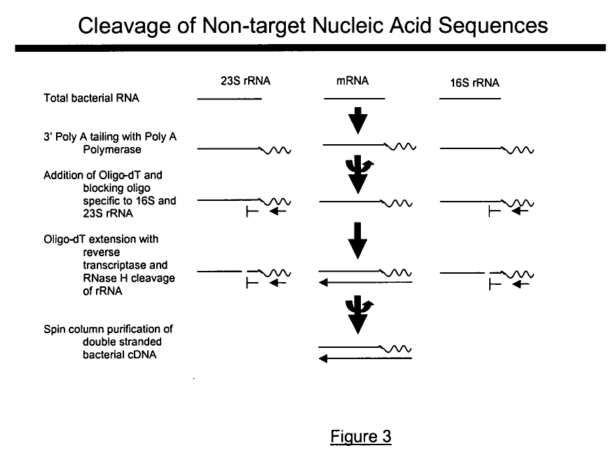

However, RNase H mediated truncation of cDNA synthesis is not an exclusive mechanism for blocking reverse transcription. Alpha-oligonucleotides bound, in parallel orientation, to RNA template immediately downstream of the primer oligo arrested the cDNA synthesis by Avian Myeloblastosis Virus-Reverse Transcriptase (AMV-RT) through an RNase H-independent mechanism. Therefore, reverse transcription inhibition through RNase H independent mechanism such as the blockage of cDNA synthesis from the primer oligo-dT due to the hybridization of the added non-extendable oligonucleotides to the rRNA templates downstream of the primer is not beyond the scope of this invention (FIG. 3). Nuclease-resistant modified oligonucleotides such as a) peptide nucleic acids (PNAs) and b) phosphorothioate oligonucleotides have been used to demonstrate the site-specific termination of transcription at a PNA-RNA complex by RT and sequence-dependent blockage of reverse transcription by RNase H independent mechanism. Certain modified oligonucleotides such as 2′-O-Alkyl oligoribonucleotides with their higher affinity for RNA templates are known to prevent cDNA synthesis by AMV-RT irrespective of where they bind to the template and even with one or two mismatches, either adjacent to or downstream of the primer. Physical blocking of reverse transcription is also possible when oligonucleotides are covalently linked to the complementary sequence on the target, a technique that might be used with strand-displacing polymerases.

In an analogous system, sequence-specific inhibition of PCR and inhibition of PCR by non-extendable oligonucleotides bound to select rDNA targets was accomplished using a mutant Taq DNA polymerase lacking its 5′ exonuclease activity these species-specific non-extendable oligonucleotides hybridized to E. coli and B. subtilis rDNA inter-primer region inhibiting PCR while allowing unrestricted amplification of the N. gonorrohea rDNA, which had non-complementary sequences to the non-extendable oligonucleotides. Similarly, reverse transcriptase's which are DNA polymerases capable of DNA synthesis using RNA templates and generally lacking in 5′ exonuclease activity and strand displacing activity may fail to extend primer beyond the non-extendable oligonucleotides bound downstream of priming regions. Therefore, one could reasonably surmise the use of non-extendable oligonucleotides in Reverse Transcription-Polymerase Chain Reaction (RT-PCR), PCR or primer extension techniques such as Nucleic Acid Sequence Based Amplification (NASBA) to selectively block undesirable targets and preferentially amplify a target in order to enrich for that target. One such application is to use non-extendable oligonucleotide to physically interrupt the extension of the cDNA initiated from rRNA or rDNA bound primer while presenting no such physical hurdle for the cDNA synthesis by DNA polymerases from unblocked mRNA templates or their complements.

The present invention embodies the use of reverse transcriptase's (RT) that are preferably, but not limited to, functional between 30 and 60 degrees Celsius. Suitable reverse transcriptase's preferably include, but are not limited to, commonly available (Avian Myeloblastosis Virus) AMV-RT, (Moloney Murine Leukemia Virus) MMLV-RT, (Rouse Sarcoma Virus) RSV-RT, (Human Immuno-deficiency Virus) HIV1-RT, HIV2-RT etc. In further embodiments of the present invention, it may be preferable to add additional RNase H purified from E. coli, although some reverse transcriptase's have an inherent RNase H activity that act independently of the polymerization activity. Added RNase H may be necessary where RT without RNase H activity is used or where the inherent RNase H is inhibited by some reagents or components (due to competition between oligo-dT and rRNA/rDNA sequence specific oligos for RT) of the kit or is insufficient to accomplish the task of degrading the RNA template bound to its complement such as an exogenously added modified or unmodified rRNA/rDNA specific oligo. It is also within the scope of the invention to use rRNA specific oligos conjugated with an RNase H for the purposes of degrading its RNA complement upon hybridization to it. Such a strategy has been employed previously to cleave hepatitis B viral messenger RNA.

In certain aspects of the present invention, dNTPs used during the cDNA synthesis may be labeled with fluorescent, radioactive or non-radioactive dyes. Such labeling of cDNA during reverse transcription of DNA synthesis is well known to one skilled in the art. One such labeling technique to generate a cDNA probe with Fluorescent dyes is through reverse transcription of targeted RNA in the presence of Cy3- or Cy5-dCTP. Labeled nucleic acid molecules are especially useful in the study of analyses of genes expressed by a bacterium genome, a bacterial community, host-bacterial cell infection. Gene expression analyses are well known to one skilled in the art and routine analyses are performed by hybridizing pooled labeled mRNA or cDNA libraries to oligonucleotide-arrays.

Following the selective conversion of the bacterial mRNA into their cDNA counterparts, a number of strategies could be used to separate the bacterial cDNA either as the single stranded poly-A tailed molecules or cDNA molecules or the double stranded cDNA-mRNA molecules depending upon the need. One approach could be to degrade all RNA molecules with RNase H or other RNase's such as RNase A and recover only the higher molecular weight single stranded cDNA by any number of commercially available ssDNA purification columns such as QiaPrep (Qiagen, Calif.). Alternatively, the post reverse transcription product of cDNA-mRNA hybrid molecules may also be separated. An easy way to recover these molecules is to use the spin-columns such as the DNA Clean and Concentrator Column (DCC™) commercially made available by Zymoresearch (Orange, Calif.) that purifies the reaction mixture disclosed in the preceding description of the invention. These spin columns remove any unused dNTPs, buffers, reagents and oligonucleotide fragments etc. Such a spin column may even be supplied with the bacterial cDNA synthesis kit envisioned in this invention. In the event oligo-dT sequences derivatized with magnetic beads are used in cDNA synthesis, the bacterial mRNA molecules bound to these oligos can be separated by magnetic bead separation technique.

In one particular embodiment of the present invention thermostable RT, PAP and RNase H may be used. The enzymes of the present invention are intended for use between the ambient temperature to 65 degrees Celsius.

What is claimed is:

1. A method for enriching, isolating, separating or purifying a targeted nucleic acid molecule (bacterial mRNA) from a sample through selective full-length reverse transcription (primer extension) comprising
a) Incubating with a 3′ tailing enzyme that tails of all nucleic acid (RNA) molecules
b) Incubating the sample with a DNA polymerase (reverse transcriptase) and
i. oligonucleotide capable of primer extension by hybridizing to the nucleotide tail of targeted and non-targeted nucleic acid molecules
ii. at least one another non-extendable oligonucleotide capable of hybridizing to at least one non-target nucleic acid molecule (rRNA) capable of blocking or inhibiting primer extension
c) Purification of the targeted nucleic acid molecule (mRNA)

2. A method to enrich, isolate or separate or purify a targeted nucleic acid molecule (bacterial mRNA) from a sample comprising
a) Incubating with at least one non-tailed oligonucleotide derivatized with magnetic bead capable of binding to at least one non-target nucleic acid molecule (rRNA) or its complement
b) Purification of targeted nucleic acid molecule (mRNA) by the separation of the rRNA bound to oligonucleotide derivatized with a magnetic bead using a magnet.

3. The method of claim 1, wherein the targeted nucleic acid molecule includes a bacterial mRNA or a eukaryotic mRNA.

4. The method of claim 1, wherein the non-targeted nucleic acid molecule is a prokaryotic small subunit rRNA (16S) or large subunit rRNA (23S) and/or eukaryotic small subunit rRNA (17S and 18S) or large subunit rRNA (28S) or 5S RNA

5. The method of claim 1, wherein the sample comprises of eukaryotic or prokaryotic nucleic acid molecules.

6. The method of claim 1, wherein the tailing enzyme is preferably a poly A polymerase

7. The method of claim 1, wherein the DNA polymerase is preferably a reverse transcriptase, said reverse transcriptase having both DNA-dependent DNA polymerase and an RNA-dependent DNA polymerase activity.

8. The method of claim 1, wherein the 3′ tail to the nucleic acid is added by a DNA or RNA ligase and wherein the 3′ tail comprises of a promoter region for T7 RNA polymerase to bind and initiate primer extension

9. The method of claim 1, wherein the oligonucleotide capable of primer extension is complementary to the 3′ tail, is an oligo-dT, an oligo-dN which is biotinylated, or derivatized with magnetic bead

10. The method of claim 9, wherein the oligonucleotide capable of primer extension comprises a bead comprising of a solid support made of cellulose, latex, silica, plastic, polystyrene, nylon, nitrocellulose, polyvinylchloride, styrene-divinylbenzene, polymethacrylate, magnetized material or glass.

11. The method of claim 1, wherein the primer extension (reverse transcription) uses labeled nucleotides.

12. The method of claim 1, wherein the non-extendable oligonucleotide hybridizes anywhere on the non-target molecule or preferably within 250, 150, 100 or 50 nucleotides from the first nucleotide added by a tailing enzyme

13. The method of claim 1, wherein the non-extendable oligonucleotide is modified by a chemical modification, modification, by a phosphorothioate bond, by a peptide bond, or by a covalent linkage with a RNase H

14. The method of claim 1, wherein the blocking of primer extension on a non-target nucleic acid molecule by a non-extendable oligonucleotide is through cleavage of the template by a RNase H, wherein said RNase H activity is supplied by a reverse transcriptase or RNase H.

15. The method of claim 1, wherein the inhibition of primer extension on a non-target nucleic acid molecule by a non-extendable oligonucleotide is through physical blocking of primer extended by a DNA polymerase

16. The method of claim 7, wherein the reverse transcriptase has a non-strand displacing property

17. The method of claim 1, wherein the purification of the target nucleic acid is through a spin column or precipitation

18. The method of claim 1, wherein the separation of the target nucleic acid synthesized with an oligo-dT or oligo-dN derivatized with a magnetic bead is through purification by a magnetic stand

19. The method of claim 1, wherein the purification of the target nucleic acid is as a single strand molecule and is preceded by the degradation of all RNA by RNases

20. The method of claim 1, wherein the purification of the target nucleic acid is as a double strand molecule such as DNA-RNA hybrid or DNA-DNA hybrid

21. A kit, in a suitable container, comprising of oligo-dT, non-extendable oligonucleotides, reverse transcriptase, RNase H, poly A polymerase and corresponding buffers, NTPs, dNTPs are included

22. The method of claim 1, further comprising of generating cDNA libraries, cDNA libraries in a vector capable of propagating in a live host, cDNA libraries in a vector capable of propagating in vivo, cDNA libraries in a vector capable of propagating in vitro

23. The method of claim 1, further comprising of constructing a cDNA array in solution or on solid support with the purpose to interrogate or identify specific metabolic state, infectious agent in clinical and other diagnostic applications

24. The method of claim 1, wherein the sample is obtained from a bacterial community or bacterial isolated from a surface soil, sub surface soil or a deep subsurface soil, a host-bacterial infection, or a biofilm, mouth, intestine, fecal matter

25. The method of claim 1, wherein the sample is preserved, frozen or fixed tissue, organ or body fluid

26. The method of claim 1, wherein the nucleic acid molecules for 3′ tailing are RNA and wherein the added 3′ tail is at least 10 nucleotides

27. The method of claim 1, wherein the targeted nucleic acid for selective primer extension (reverse transcription) is a mRNA, degraded or full length.

28. The method of claim 1, wherein the non-extendable oligonucleotide is at least 10, more preferably at least 15 nucleotides

29. The method of claim 1, wherein the non-extendable oligonucleotides include nucleic acid sequences which are capable of binding any region of the eukaryotic or prokaryotic small or large subunit ribosomal RNA, nucleic acid sequences that are available in the ribosomal database projects or nucleic acid sequences with one, two or three mismatches to their complementary sequences on the non-targeted molecule such as rRNA.
